(19) United States
(12) Patent Application Publication
(10) Pub. No.: US 2021/0137381 A1
(43) Pub. Date: May 13, 2021

(54) ELECTRODE UNIT FOR MEASURING ELECTROPHYSIOLOGICAL SIGNALS

(71) Applicants: Wenzan ZHANG, SHANGHAI (CN); Xun JIN, Shanghai (CN); Leilei ZHANG, Shanghai (CN)

(72) Inventors: Wenzan ZHANG, SHANGHAI (CN); Xun JIN, Shanghai (CN); Leilei ZHANG, Shanghai (CN)

(21) Appl. No.: 16/710,696

(22) Filed: Dec. 11, 2019

(30) Foreign Application Priority Data
Nov. 8, 2019 (CN) 201911088600.7

(51) Int. Cl. A61B 5/00; A61B 5/0408

(52) U.S. Cl. CPC A61B 5/0006; A61B 5/0478; A61B 5/04085

(57) ABSTRACT

An electrode unit for measuring physiological electrical activity in a subject is provided. The electrode unit comprises a base material, an electrode, a connector, a conductive ribbon, a space limiting module, and a film. The base material includes a first base material layer and a second base material layer. The electrode is inserted in an opening of the first base material layer. The conductive ribbon is placed on the first base material layer. The conductive ribbon connects the electrode and the connector. The connector is placed inside the space limiting module. The space limiting module is placed inside an opening of the film. The second base material layer is placed on top of the conductive ribbon. The film is placed over the first base material layer and the second base material layer.

### TECHNICAL FIELD

The present disclosure relates to an electrode unit, in particular relates to an electrode unit for measuring electrophysiological signals of a subject.

### BACKGROUND

The collection of human physiological electrical signals can directly reflect the health condition of the human body. Electrocardiogram (ECG) information collection equipment is widely used in clinical activities to monitor physical condition of a patient. From the abnormal changes in the ECG information, medical staffs can discover the abnormality of the patient in a timely manner, which can help to make a diagnosis, and treat the patient.

In order to prevent cross infection, a disposable Ag/AgCl ECG electrode is often used, which is directly attached to the skin of the patient's body during use. The disposable Ag/AgCl ECG electrode is affixed with a convex electrode part, which forms an inserting connection with the electrode wires on an ECG state analysis device. However, such a convex electrode structure may result in lack of compactness of the ECG. In addition, the convex electrode part may be made of metal materials with a large mass, which may lead to a displacement of electrode with the patient's body movement. It is not suitable for physiological research of athletes or for some work that requires diagnosis with body movements. Therefore, in a new design of an electrode unit to collect ECG signal, the structure of electrode needs to be simplified as much as possible, and the electrode unit may include as many ECG electrodes as possible, which is conveniently to use. At the same time, light-weight materials may be used to increase comfort, air permeability and waterproofness, which is convenient for long-term monitoring.

### SUMMARY

The technical problems to be solved by this present disclosure are to provide a design of ECG electrode, with features including: an elasticity light weight fabric as a base material; directly adhering to skin through special adhesive gel for skin, to increase the adhesion to skin; reducing the mass of the measuring electrode and reducing motion artifacts.

An electrode unit for measuring physiological electrical activity in a subject is provided. The electrode unit comprises a base material, an electrode, a connector, a conductive ribbon, a space limiting module, and a film.

In one embodiment, the base material includes a first base material layer and a second base material layer.

In one embodiment, the electrode is inserted in an opening of the first base material layer. The conductive ribbon is placed on the first base material layer.

In one embodiment, the conductive ribbon connects the electrode and the connector. The connector is placed inside the space limiting module.

In one embodiment, the space limiting module is placed inside an opening of the film.

In one embodiment, the second base material layer is placed on top of the conductive ribbon.

In one embodiment, the film is placed over the first base material layer and the second base material layer.

In one embodiment, the electrode unit further comprises an adhesive tape which is employed in a gap between the connector and the space limiting module.

In one embodiment, the electrode unit further comprises a first release paper and a second release paper.

In one embodiment, the base material includes an elastic material of non-woven fabric, cotton, polyester or nylon.

In one embodiment, the elasticity of the base material is equivalent to the elasticity of human muscle.

In one embodiment, a bottom surface of the base material is coated with an adhesive gel that is specific for human skin.

In one embodiment, the adhesive gel specific for human skin includes a menthol component for cooling, anti-itching, antibacterial and anti-inflammatory effects.

In one embodiment, the electrode includes a conductive paste, a conductive gel or a composite dry electrode including CNT-PDMS or CNT-Ag-PDMS.

In one embodiment, the electrode includes a menthol component for cooling, anti-itching, antibacterial and anti-inflammatory effects.

In one embodiment, the connector includes a conductive gel.

In one embodiment, the conductive ribbon includes a conductive wire woven by a metal wire or a conductive ribbon woven from polyester fiber.

In one embodiment, the conductive ribbon includes the same elasticity as the base material.

In one embodiment, the space limiting module includes a biocompatible polymeric material. The biocompatible polymeric material includes one or more of silica gel, thermoplastic polyurethane (TPU) and polyethylene terephthalate (PET).

In one embodiment, the film is made of a membrane.

In one embodiment, the film includes properties of extensibility, waterproof property and breathability.

In one embodiment, the adhesive tape, the connector and the space limiting module, together perform a function of connecting the electrode unit and the ECG signal acquisition device.

### BRIEF DESCRIPTION OF THE DRAWINGS

Features of the subject technology are set forth in the appended claims. However, for purpose of explanation, several embodiments of the subject technology are set forth in the following figures.

FIG. 1 is a schematic structural diagram of an exemplary electrode unit according to an embodiment of the present disclosure.

FIG. 2 is a structural cross-sectional view of the electrode unit of FIG. 1 according to an embodiment of the present disclosure.

FIG. 3 is a diagram showing an assembly of the electrode unit of FIG. 1 according to an embodiment of the present disclosure.

FIG. 4 is a diagram schematically showing the electrode unit of FIG. 1 placed on a subject according to an embodiment of the present disclosure.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced without these specific details. In some instances, structures and components are shown in block diagram form in order to avoid obscuring the concepts of the subject technology.

It is to be understood that aspects of the present disclosure will be described in terms of a given illustrative architecture; however, other architectures, structures, materials and process features and steps can be varied within the scope of aspects of the present disclosure.

It will also be understood that when an element such as a layer, region or substrate is referred to as being “on” or “over” another element, it can be directly on the other element or intervening elements can also be present. In contrast, when an element is referred to as being “directly on” or “directly over” another element, there are no intervening elements present.

It will also be understood that when an element is referred to as being “connected” or “coupled” to another element, it can be directly connected or coupled to the other element or intervening elements can be present. In contrast, when an element is referred to as being “directly connected” or “directly coupled” to another element, there are no intervening elements present.

The present embodiments can include a design for a medical device, which may include multiple features or combinations of features. Some or all features may or may not be present on the devices in accordance with embodiments of the present disclosure.

Reference in the specification to “one embodiment” or “an embodiment”, as well as other variations thereof, means that a particular feature, structure, characteristic, and so forth described in connection with the embodiment is included in at least one embodiment. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This can be extended, as readily apparent by one of ordinary skill in this and related arts, for as many items listed.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements and/or components, but do not preclude the presence or addition of one or more other features, integers, steps, operations, elements, components and/or groups thereof.

Spatially relative terms, such as “beneath”, “below”, “lower”, “above”, “upper”, and the like, can be used herein for ease of description to describe one element's or feature's relationship to another element (s) or feature (s) as illustrated in the FIG. 1t will be understood that the spatially relative terms are intended to encompass different orientations of the device in use or operation in addition to the orientation depicted in the FIGS. For example, if the device in the FIGS. is turned over, elements described as “below” or “beneath” other elements or features would then be oriented “above” the other elements or features. Thus, the term “below” can encompass both an orientation of above and below. The device can be otherwise oriented (rotated 90 degrees or at other orientations), and the spatially relative descriptors used herein can be interpreted accordingly. In addition, it will also be understood that when a layer is referred to as being “between” two layers, it can be the only layer between the two layers, or one or more intervening layers can also be present.

It will be understood that, although the terms first, second, etc. can be used herein to describe various elements, these elements should not be limited by these terms. These terms are only used to distinguish one element from another element. Thus, a first element discussed below could be termed a second element without departing from the scope of the present disclosure.

The system and methods of the present embodiments may also be employed on animals, models and other non-living substrates, such as, for example, in training, testing and demonstration.

It is to be understood that the present embodiments are not limited to the specific devices, methods, conditions or parameters described and/or shown herein, and that the terminology used herein is for the purpose of describing particular embodiments by way of example only and is not intended to be limiting. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

FIG. 1 is a schematic structural diagram of an exemplary electrode unit according to an embodiment of the present disclosure.

As illustrated in FIG. 1, an electrode unit 100 comprises a base material 101, an electrode 102, a connector 103, a conductive ribbon 104, a space limiting module 105, an adhesive tape 106, and a film 107.

According to one embodiment of the present disclosure, the base material 101 may be an elastic light weight patch woven of non-woven fabric, cotton, polyester and nylon produced by mechanical mesh weaving technology. The base material 101 may match the human skin texture, which results in smooth affix to human skin. The elasticity of the base material 101 may be equivalent to the elasticity of human muscle. There may be scientifically designed holes in the base material 101 to provide an excellent breathability. The base material 101 may include features of good air permeability, good elasticity and/or soft skin touch, which improves the user's comfort. A special adhesive gel for human skin may be coated on a bottom surface of the base material 101, which can ensure that the electrode unit 100 is securely affixed to human skin. In some embodiments, the thickness of the base material 101 may be, for example, less than 0.5 mm. Thus, the base material 101 may include properties, such as softness and/or light weight, resulting in a great user experience. For example, the thickness of the base material 101 may be set to approximately 0.4 mm. In some embodiments, the special adhesive gel for human skin may be added with a menthol component to achieve a cooling, anti-itching, antibacterial and anti-inflammatory effect.

According to one embodiment of the present disclosure, the electrode 102 may include an ECG electrode. The electrode unit 100 may comprise two electrodes 102, which may be symmetrically distributed on both sides of the space limiting module 105, so that the ECG signal can be more stable. In some embodiments, the electrode unit 100 may include one, two, three of the electrode 102, or more. The set of two electrodes 102 may be configured to collect ECG signals by contacting human skin. The electrode 102 may be a conductive gel cut into a circle, and the diameter of the circular conductive gel may be approximately 5 mm to 20 mm. In some embodiments, the conductive gel may be formed by a water-soluble or hydrophilic polymer through certain chemical crosslinking or physical crosslinking, which may avoid any discomfort to human body and improve the user's experience. In some embodiments, a menthol component may be further added to the conductive gel for cooling, anti-itching, antibacterial and anti-inflammatory effects. In some embodiments, the electrode 102 may also be made of a conductive paste, a CNT-PDMS composite dry electrode material, a CNT-Ag-PDMS composite dry electrode material, or other materials.

The connector 103 may include an adhesive connector. The connector 103 may be configured to connect with an ECG signal acquisition device and transmit the ECG signals collected by the electrode 102. In some embodiments, the electrode unit 100 may include one, two, three of the connector 103, or more. The number of the connector 103 may coincide with the number of the electrode 102. The material of the connector 103 may be a medical conductive gel, which may be viscous and can be tightly affixed to a metal conductive sheet from the ECG signal acquisition device. The shape of the connector 103 may be circular, and the diameter of the connector 103 may be approximately 5 mm to 20 mm. The plurality of the connector 103 may be separated from each other and evenly distributed inside the space limiting module 105. The connector 103 may be connected with the electrode 102 via the conductive ribbon 104.

The conductive ribbon 104 may include an elastic conductive ribbon. The conductive ribbon 104 may be a conductive wire woven by a metal wire such as a conductive silver nanowire and/or a conductive ribbon woven from polyester. The elasticity of the conductive ribbon 104 may be equivalent to the elasticity of the base material 101, so that the measurement stability of the electrode unit 100 remains when human muscles are stretched. In some embodiments, the electrode unit 100 may include one, two, three of the conductive ribbon 104, or more. The number of the conductive ribbon 104 may coincide with the number of the electrode 102.

According to one embodiment of the present disclosure, the material of the space limiting module 105 may include polymer, such as for example, polyethylene terephthalate (PET), which may be a milky white or light yellow, highly crystalline polymer with a smooth and glossy surface. The space limiting module 105 may have a high creep resistance, fatigue resistance and friction resistance. The space limiting module 105 may be an excellent barrier against air, water, oil. The space limiting module 105 may also have a good dimensional stability. In some embodiments, the material of the space limiting module 105 may further include a biocompatible polymeric material such as silica gel or thermoplastic polyurethane (TPU). The space limiting module 105 may be configured to secure the contact of the connector 103 and a connecting media from the ECG signal acquisition device to perform a stable signal acquisition. In addition to the space limiting module 105, the adhesive tape 106 may be employed in the gap between the surface of the connector 103 and the space limiting module 105, so that the electrode unit 100 and the ECG signal acquisition device can be connected tightly and stably, thereby avoiding one or more displacements. In some embodiments, the adhesive tape 106 may be a double-sided tape. In some embodiments, the adhesive tape 106, the connector 103 and the space limiting module 105, together perform the function of connecting the electrode unit 100 and the ECG signal acquisition device, so that ECG signal acquisition is more stable.

According to one embodiment of the present disclosure, the film 107 may include an elastic breathable waterproof film. The film 107 may be employed to cover the upper surface and the edge of the electrode unit 100, to provide a waterproofing feature. The space limiting module 105 may be exposed through an opening in the film 107. In some embodiments, the space limiting module 105 and the connector 103 can be accessed for connecting with the ECG signal acquisition device. The film 107 may be made of a membrane, for example, a polyurethane waterproofing membrane, which is a non-toxic and harmless highly elastic and environmentally friendly material. The film 107 may have properties including a high extensibility, a waterproof property and breathability. In some embodiments, the thickness of the film 107 may be small, for example, approximately 0.05 mm. In some embodiments, the film 107 may have an excellent waterproofing and an excellent breathability. The film 107 can withstand water pressure over 1000 mm. The film 107 may have a wet steam permeability of approximately 500-600 g/m2/24 h. In some embodiments, human sweat can penetrate freely through the film 107. By using the high elasticity, excellent waterproofing and high breathability materials, the electrode unit 100 can perform ECG signal acquisition during one or more activities, such as including sport activities and bathing, as well as a long-term dynamic measurement of ECG signals.

In some embodiments, the components of the electrode unit 100 may all be made of elastic materials. Thus, the electrical resistance value between the skin of a subject and the electrode unit 100 may remain about the same under different motion states of a subject of the electrode unit 100. In some embodiments, the electrical resistance between the electrode 102 and the connector 103 of the electrode unit 100 may remain about the same when a subject wearing the electrode unit 100 is bathing. In some embodiments, the electrical resistance between the electrode 102 and the connector 103 of the electrode unit 100 may show no significant change after being used for 24 hours. The electrical resistance between the electrode 102 and the connector 103 of the electrode unit 100 may also show no significant change under chest expansion or stationary state of a subject wearing the electrode unit 100.

FIG. 2 is a structural cross-sectional view of the electrode unit of FIG. 1 according to an embodiment of the present disclosure.

In one embodiment, FIG. 2 illustrates a structural cross-sectional view of the electrode unit 100 along the center line a-a′ as shown in FIG. 1. As illustrated in FIG. 2, an operational relationship between all components of one embodiment of the electrode unit 100 is presented. In FIG. 2, the electrode unit 100 includes two layers of the base material 101, including a first base material layer 101A, and a second base material layer 101B, the electrode 102, the connector 103, the conductive ribbon 104, the space limiting module 105, the adhesive tape 106, the film 107, and two pieces of release paper, including a first release paper 108A and a second release paper 108B.

According to one embodiment of the present disclosure, the electrode unit 100 is affixed to the skin of a subject using the first base material layer 101A. A high-quality medical pressure sensitive adhesive may be coated using a breathable coating process on the bottom surface of the first base material layer 101A. In some embodiments, the first base material layer 101A may maintain viscosity for 3 to 5 days, even under a sweating condition of a subject using the electrode unit 100. The first base material layer 101A may include one or more through holes, which may be set far apart and evenly distributed in the first base material layer 101A. In some embodiments, the through holes may be circular or square. As shown in FIG. 2, the electrode 102 may be placed inside the through hole in the first base material layer 101A to contact skin of a subject, and may be configured to collect ECG signals from a subject, such as a human body. The diameter of the through hole in the first base material layer 101A may be approximately 4 mm to 19 mm, which may be slightly smaller than the diameter of the electrode 102. Thus, the electrode 102 may be firmly inserted in the first base material layer 101A without any displacement. The conductive ribbon 104 may be placed on the first base material layer 101A and may be affixed by binding or sewing. The second base material layer 101B may be placed on a portion of the conductive ribbon 104 and a portion of the electrode 102. The upper surface of the conductive ribbon 104 and the upper surface of the electrode 102 may be adhered to the bottom surface of the second base material layer 101B. Thus, the conductive ribbon 104, the first base material layer 101A and the second base material layer 101B can be stably affixed, which results in a visual integrity of the electrode unit 100. The conductive ribbon 104 may be configured to electrically connect the electrode 102 and the connector 103. In some embodiments, one end of the conductive ribbon 104 may be placed on the electrode 102. In some embodiments, the remaining end of the conductive ribbon 104 may connect to an end of the connector 103. The connector 103 may cover the remaining end of the conductive ribbon 104.

The space limiting module 105 may connect with the second base material layer 101B and may be placed on the conductive ribbon 104. One or more connectors 103 may be evenly distributed inside the space limiting module 105. As shown in FIG. 2, for example, two connectors 103 may be distributed inside a different end of the space limiting module 105.

In some embodiments, the connector 103 may connect with the conductive ribbon 104 to establish an electrical connection. In one or more gaps between the connector 103 and the space limiting module 105, the adhesive tape 106 may be used to secure the attachment between the ECG signal acquisition device and the connector 103. The film 107 may be formed over the second base material layer 101B, covering the upper surface and the edge of the electrode unit 100. The space limiting module 105, as well as the connector 103 and the adhesive tape 106, may be exposed via an opening in the film 107 for direct access. In some embodiments, the opening in the second base material layer 101B and the opening in the film 107 may be aligned. The film 107 may bind with the second base material layer 101B directly by adhesive gel. The first release paper 108A may cover the bottom of the electrode unit 100 as well as the edge of the film 107, and the second release paper 108B may cover the exposed surface inside the space limiting module 105. The first release paper 108A and the second release paper 108B may have openings or protrusions to facilitate the easiness of peeling off of the first release paper 108A and/or the second release paper 108B in usage. The first release paper 108A and the second release paper 108B may perform as protection agents of the electrode unit 100 while the electrode unit 100 is not being used.

The electrode unit 100 provided by the present disclosure may have one or more advantages such as simple structure, light weight and convenience of use. This design of present disclosure can increase the adhesion tightness with skin and reduce motion artifacts; it can perform the ECG monitoring during exercise and bathing, and can also perform long-term dynamic ECG monitoring.

FIG. 3 is a diagram showing an assembly of the electrode unit 100 according to the present disclosure.

As illustrated in FIG. 3, the electrode unit 100 comprises two layers of the base material 101, including the first base material layer 101A, and the second base material layer 101B, and the film 107. As can be seen in FIG. 3, when assembled, the components of the electrode unit 100 may be formed between the film 107 and the first base material layer 101A, including the electrode 102, the connector 103, the conductive ribbon 104, the space limiting module 105 and the adhesive tape 106. The electrode 102 may be inserted in a through hole of the first base material layer 101A. The conductive ribbon 104 may be placed between the first base material layer 101A and the second base material layer 101B. In some embodiments, one end of the conductive ribbon 104 may be placed on the electrode 102. In some embodiments, the remaining end of the conductive ribbon 104 may connect to an end of the connector 103. The connector 103 may cover the remaining end of the conductive ribbon 104. In some embodiments, the conductive ribbon 104 may be configured to electrically connect the electrode 102 and the connector 103. The connector 103 may locate inside the space limiting module 105. The adhesive tape 106 may be employed in the gap between the connector 103 and the space limiting module 105. Thus, the electrode unit 100 and the ECG signal acquisition device can be connected tightly and stably, to avoid any displacement. The space limiting module 105, as well as the connector 103 and the adhesive tape 106 inside the space limiting module 105, may be exposed via the opening in the film 107 for direct access. The film 107 may enable the electrode unit 100 to be used in bathing. For a convenient operation, the first release paper 108A may cover the bottom surface of the electrode unit 100, and the second release paper 108B may cover the exposed surface inside the space limiting module 105. The electrode unit 100 can be used after the first release paper 108A and the second release paper 108B are peeled off.

FIG. 4 is a diagram schematically showing the electrode unit of FIG. 1 placed on a subject according to an embodiment of the present disclosure.

As illustrated in FIG. 4, the electrode unit 100 is affixed to a left chest of a subject, such as a human body, while the subject is in a stationary state 400. In some embodiments, the electrode unit 100 may be affixed to the left chest of the subject while the subject is having a chest expansion movement. In some embodiments, the electrode unit 100 may have approximately the same amount of stretching as human muscle stretches. The long-term monitoring of human ECG signals may be performed by connecting the electrode unit 100 with a corresponding ECG information monitoring equipment. When human skin is deformed, for example, being stretched comparing to stationary state, the electrode unit 100 may be stretched to the approximately same degree.

In some embodiments, the electrode unit 100 of the present disclosure can be used as follows: firstly, the first release paper 108A at the bottom of the electrode unit 100 is peeled off. Secondly, the electrode unit 100 is adhered directly onto a subject, such as human skin, for example, the skin of left chest of a human (e.g., as shown in FIG. 4). Thirdly, the second release paper 108B inside the space limiting module 105 is peeled off. Then, the connector 103 is connected with a conductive metal sheet of the ECG signal acquisition device with the assistance of the adhesive tape 106, to quickly perform ECG signal monitoring. The long-term monitoring of human ECG signals may be performed by connecting the electrode unit 100 with an ECG information monitoring equipment.

In some embodiments, the ECG signal acquisition device may comprise one or more components such as, including a controller, a signal processing hardware, a data storage hardware, a communications hardware and a user interface component. In some embodiments, the ECG information monitoring equipment may comprise suitably configured hardware and/or software components for processing signals from the electrode unit 100 and for generating corresponding ECG waveforms for display. These components may be configured to provide particular functionality using suitably coded software.

Although the present disclosure is disclosed in the preferred embodiments above, it is not intended to limit the present disclosure. Any person skilled in this field can make possible changes and modifications without departing from the spirit and scope of this present disclosure. Therefore, any modification, equivalent change and polish made to the above disclosure according to the essence of the technology of the present disclosure without departing from the content of the technical scheme of the present disclosure are within the scope of protection defined in the claims of the present disclosure.

What is claimed is:

1. An electrode unit for measuring physiological electrical activity in a subject, the electrode unit comprising:
a base material;
an electrode;
a connector;
a conductive ribbon;
a space limiting module; and
a film,
wherein the base material includes a first base material layer and a second base material layer,
wherein the electrode is inserted in an opening of the first base material layer,
wherein the conductive ribbon is placed on the first base material layer,
wherein the conductive ribbon connects the electrode and the connector,
wherein the connector is placed inside the space limiting module,
wherein the space limiting module is placed inside an opening of the film,
wherein the second base material layer is placed on top of the conductive ribbon, and
wherein the film is placed over the first base material layer and the second base material layer.

2. The electrode unit of claim 1, further comprising an adhesive tape which is employed in a gap between the connector and the space limiting module.

3. The electrode unit of claim 1, further comprising a first release paper and a second release paper.

4. The electrode unit of claim 1, wherein the base material includes an elastic material of non-woven fabric, cotton, polyester or nylon.

5. The electrode unit of claim 1, wherein the elasticity of the base material is equivalent to the elasticity of human muscle.

6. The electrode unit of claim 1, wherein a bottom surface of the base material is coated with an adhesive gel that is specific for human skin.

7. The electrode unit of claim 6, wherein the adhesive gel specific for human skin includes a menthol component.

8. The electrode unit of claim 1, wherein the electrode includes a conductive paste, a conductive gel or a composite dry electrode, including CNT-PDMS or CNT-Ag-PDMS.

9. The electrode unit of claim 8, wherein the conductive gel includes a menthol component.

10. The electrode unit of claim 1, wherein the connector includes a conductive gel.

11. The electrode unit of claim 1, wherein the conductive ribbon includes a conductive wire woven by a metal wire or a conductive ribbon woven from polyester.

12. The electrode unit of claim 1, wherein the conductive ribbon includes the same elasticity as the base material.

13. The electrode unit of claim 1, the space limiting module includes a biocompatible polymeric material, which includes one or more of silica gel, thermoplastic polyurethane (TPU) and polyethylene terephthalate (PET).

14. The electrode unit of claim 1, wherein the film is made of a membrane.

15. The electrode unit of claim 1, wherein the film includes properties of extensibility, waterproof property and breathability.

16. The electrode unit of claim 1, wherein the adhesive tape, the connector and the space limiting module, together perform a function of connecting the electrode unit and an ECG signal acquisition device.
